(19) United States
(12) Patent Application Publication
(10) Pub. No.: US 2003/0116462 A1
(43) Pub. Date: Jun. 26, 2003

(54) Pouch configuration for wrapped absorbent articles

(75) Inventors: Heather A. Sorebo, Appleton, WI (US); Wendy Benhke, Neenah, WI (US); Julie Bednarz, Neenah, WI (US); David R. Wagner, Neenah, WI (US); Robert J. Koenig, Roswell, GA (US)

Correspondence Address: STEPHEN E. BONDURA, Dority & Manning,, Attorneys at Law, P.A., P.O. Box 1449, Greenville, SC, 29602, US

(73) Assignee: KIMBERLY-CLARK WORLDWIDE, INC.

(21) Appl. No.: 10/029,127

(22) Filed: Dec. 20, 2001

(51) Int. Cl. A61F 13/15; A61F 13/20; A61L 15/00; A61B 17/06; A61B 19/02

(52) U.S. Cl. 206/438; 604/385.13

(57) ABSTRACT

A feminine care absorbent article package includes an absorbent article contained within a pouch having a panel member extending across the back of the pouch. The panel member is alternately pulled over the open top of the pouch to open and re-seal the pouch.

### BACKGROUND OF THE INVENTION

The present invention relates to individually wrapped absorbent articles such as catamenial pads. More specifically, the invention relates to a unique wrapping configuration for such products.

Absorbent feminine care articles such as sanitary napkins, panty liners, labial pads, and other types of catamenial devices are used to absorb menses and other body fluids. These absorbent products are used during a women's menstrual cycle and are primarily disposable. In addition, disposable absorbent articles are also used between menstrual cycles for light incontinence purposes. Since many of these articles are carried in a woman's purse or pocket prior to use, it is advantageous to individually wrap each article to keep it clean and sanitary. By individually packaging each absorbent article, the manufacturer can be assured that the article will not become contaminated by the contents of the user's purse, pocket, etc. Instead of individually packaged products, it is also a common practice to provide several of the products in a larger pouch referred to as a “purse pack.”

Conventionally, the pouch wrapper consists of one or more layers of a thin sheet or film of thermoplastic material, such as polyethylene, which is folded around the absorbent article and then sealed by the use of heat and/or pressure, ultrasonics, or an adhesive to form a package or pouch. The package may have a sealed side or edge that is designed to be opened by breaking or tearing the material at or adjacent the seal in order to subsequently remove the absorbent article. With some conventional package designs, a flap is provided that folds over the pouch opening and may attach to the front of the pouch with adhesive applied between the pouch and flap, or with a piece of adhesive tape. The sides of the flap are typically sealed with the sides of the pouch and must be separated prior to removing the absorbent article.

In certain situations, it is not possible or desirable for consumers to immediately dispose of used absorbent articles. For this reason, conventional packages are also typically designed so that a soiled article can be wrapped up in the opened package for later disposal. However, most conventional pouch designs are not well suited for this purpose. For example, with the pouches wherein a sealed end or side is torn open to remove the article, there is virtually no way to subsequently reseal the package once a soiled article has been placed back inside the pouch. With the flap pouch configurations, it may be possible to subsequently re-tape or re-adhere the flap to the front of the pouch, but the sides of the flap cannot be re-sealed to the pouch. The consumer that must carry used articles for later disposal are prone to the embarrassing possibility that the conventional pouches will leak or emit odor, that the used products are visible in the pouch, or even that the used articles may fall out of the pouch in the user's purse or case.

The present invention relates to an improved pouch design that is essentially resealable for storing used articles with a greater degree of reliability and confidence for the consumer.

### SUMMARY

Objects and advantages of the invention will be set forth in part in the following description, or may be obvious from the description, or may be learned through practice of the invention.

The present invention relates to an absorbent article package having a unique pouch configuration. The package is not limited to any particular type of absorbent article and may include, for example, feminine pads, liners, tampons, labial pads, and the like. The pouch may be used for individually wrapped articles, or as a purse-pack for several articles. The package includes a wrapper material configured into a pouch, wherein the absorbent article(s) is stored in the pouch. The invention is not limited to any particular type of wrapper material, which may be, for example, one or more layers of a fluid impervious film. The film layers may be vapor permeable. Various film materials are well known and widely used in the art for this purpose. The wrapper material may also be a laminate of a film and a non-woven material or other fibrous material layer.

An embodiment of an absorbent article package according to the invention includes at least one absorbent article wrapped in a wrapper material that has been configured into a pouch. The pouch includes a front surface, a back surface, closed sides and bottom, and an open or openable top side. In an open configuration of the pouch, a panel member is disposed across the back surface. This panel has a top edge contiguous with the open top side of the pouch, a bottom edge, and side edges sealed to the pouch sides. The panel has a length between its top and bottom edges so as to extend less than about half-way, desirably about one-third, down the pouch back surface.

The panel member provides an efficient and user-friendly means by which the pouch can be opened and subsequently re-sealed. The panel member is not torn, severed, or otherwise destroyed, and the pouch maintains its initial integrity upon a used article being placed therein and the pouch being re-sealed. To seal an article in the pouch, the bottom edge of the panel member is pulled by the user over the open top side of the pouch without breaking the bonds between the side edges of the panel and the pouch. The bonded portion of the pouch sides having the panel side edges bonded therewith thus essentially invert when the panel is pulled over the open top side of the pouch, and the panel defines a closure member.

To unseal the pouch to remove the initial absorbent article, a user simply pulls the panel member over the top side of the pouch onto the back surface of the pouch without breaking or tearing the bonded panel sides from the pouch sides.

In one particular embodiment of the package according to the invention, the panel member is formed of the wrapper material and is continuous with the portion of the wrapper material defining the back surface of the pouch. For example, the wrapper material may be a continuous strip of material having a first end portion folded at a first fold axis, and a second end portion folded in an opposite direction at a second fold axis. The first end portion defines the pouch front surface and the material between the first fold axis and second fold axis defines the pouch back surface. The remaining part of the second end portion defines the panel. The sides of the first portion may be sealed to the sides of the back portion by any conventional sealing method to define the closed pouch sides. The sides of the panel are sealed to the pouch sides in the same bonding operation or pass.

The invention will be described in greater detail below through reference to the attached figures and particular embodiments of the invention.

### BRIEF DESCRIPTION OF THE FIGURES

FIG. 1 is a perspective view of an individually wrapped absorbent article package according to the invention in its opened configuration;

FIG. 2 is an alternate perspective view of the package of FIG. 1 shown in its opened configuration;

FIG. 3 is a perspective view of the package of FIG. 1 shown in its closed configuration;

FIG. 4 is a cross-sectional view of the open pouch in FIG. 1 taken along the lines indicated; and

FIG. 5 is a cross-sectional view of the closed pouch in FIG. 3 taken along the lines indicated.

### DETAILED DESCRIPTION

Reference will now be made in detail to one or more embodiments of the invention, at least one example of which is shown in the drawings. The embodiments are provided by way of explanation of the invention, and not meant as a limitation of the invention. For example, features illustrated or described as part of one embodiment may be used with another embodiment to yield still a different embodiment. It is intended that the present invention include these and other modifications and variations as come within the scope and spirit of the invention.

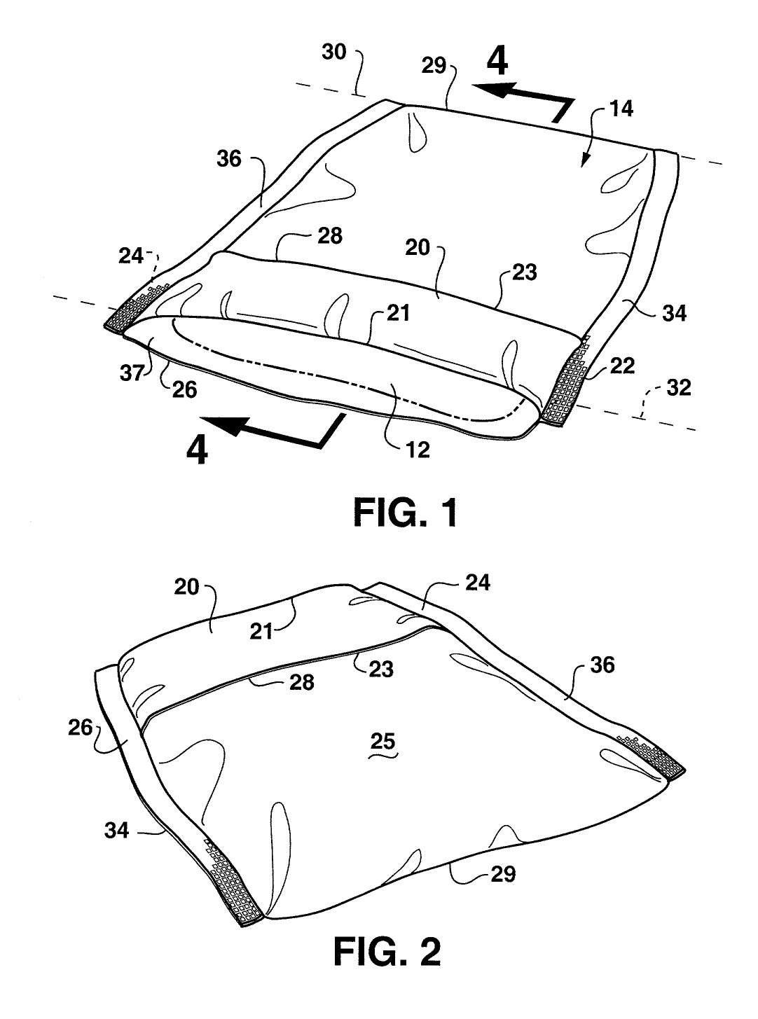

Referring to the figures, an embodiment of an individually wrapped absorbent article package 10 is generally illustrated. It should be appreciated that the invention also relates to packages, such as purse-packs, that contain more than one absorbent article. At least one absorbent article 12 (shown in dashed lines in FIG. 1) is carried in the package 10. The invention is not limited to any particular type of absorbent article. For example, the absorbent article 12, may be a catamenial device such as a sanitary napkin, a panty liner, labial pad, an incontinence pad, or any other type of absorbent article which can be used to absorb menstrual fluid, urine, body fluid, body exudates, etc. More than one type of absorbent article may be contained in the same package 10. A detailed description of such absorbent articles is not necessary for purposes of the present invention. For purposes of describing the invention only, the absorbent article 12 is shown and referred to herein as a sanitary pad or napkin. The absorbent article 12 may be folded in any desired pattern to fit in the package 10.

The package 10 includes a wrapper material, generally 14, folded and sealed into a pouch 15. The wrapper material 14 may be essentially folded around the absorbent article 12 such that the pouch 15 is formed around the article. For example, the wrapper material 14 may be an elongated rectangular piece of material having a first end 26, an opposite second end 28, and generally parallel longitudinal sides extending between the ends 26 and 28. The wrapper material 14 is folded at a first fold axis 30 such that the first end 26 is folded towards but spaced from the second end 28. The aligned longitudinal sides of the wrapper material 14 define sides 34 and 36 of the pouch 15 and the first fold axis 30 defines a closed end or bottom 29 of the pouch. A first end portion of the wrapper material between the fold axis 30 and the first end portion 26 defines a front surface 25 of the pouch. A back surface 27 is defined by the wrapper material opposite of the front surface 25 such that the interior of the pouch 15 is defined between the surfaces 25 and 27, said surfaces defining an open top side 37 of the pouch.

In an open configuration of the pouch 15 as illustrated in FIG. 1, a panel member 20 is disposed across the back surface 27. This panel 20 has a top edge 21 contiguous with the open top side 37 of the pouch 15, a bottom edge 23, and side edges 22 and 24 sealed to the pouch sides 34 and 36. The panel has a length between its top and bottom edges so as to extend less than about half-way, desirably about one-third of the way, down the pouch back surface 27. The panel member 20 may be a piece of material separate from the wrapper material 14 that is bonded to the pouch 15 at its sides 22 and 24. Desirably, the panel 20 is a continuation of the wrapper material 14 extending from a second fold axis 32 onto the back surface 27. For example, at the location of the first end 26, the wrapper material 14 is folded in an opposite direction at the second fold axis 32 such that an end portion existing between the second fold axis 32 and the second end 28 defines the panel 20. The panel side edges 22 and 24 thus align with the pouch sides 34 and 36 and are bonded therewith.

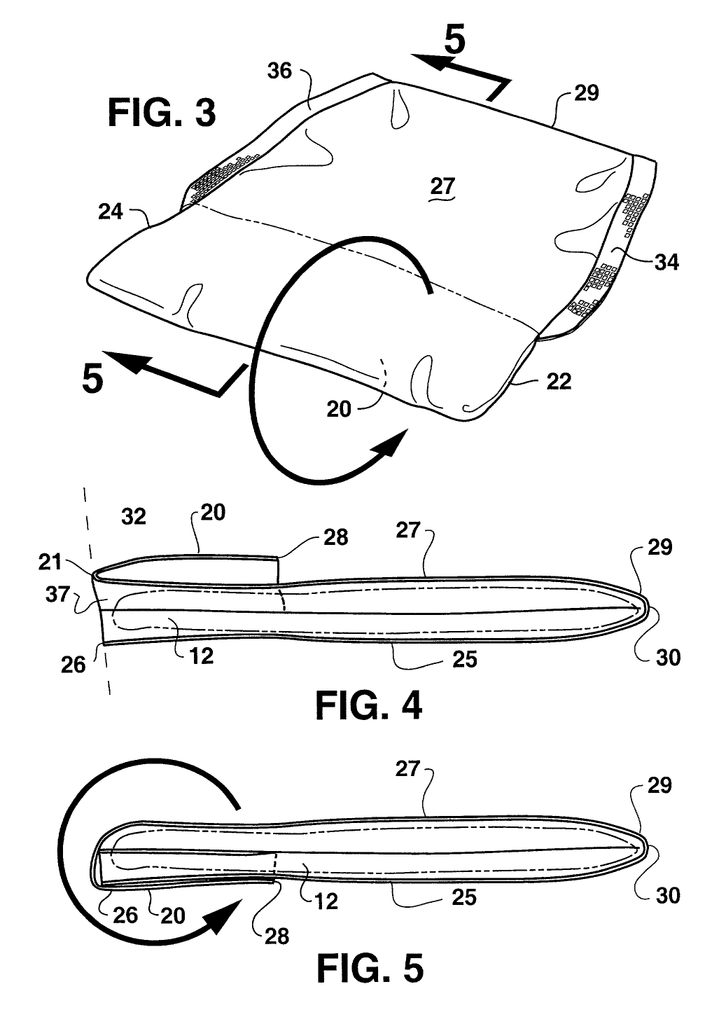

The panel member 20 provides an efficient and user-friendly closure device by which the pouch 15 can be opened and subsequently re-sealed. The panel member 20 is not torn, severed, or otherwise destroyed, and the pouch 15 maintains its initial integrity upon a used article being placed therein and the pouch being re-sealed. The pouch 15 is illustrated in an open configuration in FIGS. 1 and 2. To seal an initial or used article 12 in the pouch, the bottom edge 23 of the panel member 20 is pulled over the open top side 37of the pouch 15 as indicated by the arrows in FIGS. 3 and 5 without breaking the bonds between the side edges 22 and 24 of the panel and the sides 34 and 36 of the pouch. The bonded portion of the pouch sides 34 and 36 having the panel side edges 22 and 24 bonded therewith thus essentially invert when the panel is pulled over the open top of the pouch, as shown in FIGS. 3 and 5. The panel member 20 thus becomes a closure member.

To unseal the pouch 15 in order to remove the absorbent article 12, a user simply pulls the panel member 29 over the top side 37 of the pouch onto the back surface 27 of the pouch without breaking or tearing the bonded panel sides 22 and 24 from the pouch sides 34 and 36.

The pouch sides 34 and 36 and panel sides 22 and 24 may be bonded together by any conventional method and desirably in a single pass operation. These seals or bonds can be formed by heat, heat and pressure, pressure, adhesive, ultrasonic bonding, or other types of bonding techniques known to those skilled in the art. In a desirable embodiment, the bonds are formed with a heat/pressure embossing roll. The bonds can be made to be “permanent,” which means that the wrapper material adjacent to the seal will tear or break before the sealed layers separate.

The wrapper material 14 according to the invention may be any type of material known in the art for use in a feminine care absorbent article pouch. For example, the material may be one or more layers of fluid impervious film. The formation of films useful with the present invention is well known to those of ordinary skill in the art and need not be discussed herein in detail. One type of film that may be used is a nonporous, continuous film that, because of its molecular structure, is capable of forming a vapor-pervious barrier. Among the various polymeric films which fall into this category include films made from poly(vinyl alcohol), polyvinyl acetate, ethylene vinyl alcohol, polyurethane, ethylene methyl acrylate, and ethylene methyl acrylic acid to make them breathable. If desired, it is also possible to add fillers to the film such as, for example, calcium carbonate and titanium dioxide, to increase opacity, decrease cost, and create a breathable film if the filled film is subsequently stretched. If the film layer is not sufficiently thin, then it may be necessary to further thin the film by stretching it in an apparatus such as a machine direction orienter (MDO) unit. An MDO has a plurality of stretching rollers which progressively stretch and thin the film in the machine direction (direction of travel of the film through the machine).

Another type of film which may be useful is a microporous film. These films have a number of interconnecting voids or holes which provide pathways for the transportation of water molecules from one surface to another. The passageways are sufficiently small so that only vapors and not fluids can pass through them.

The wrapper material may also be a laminate of different materials, such as a film/nonwoven laminate.

In addition to those already mentioned, the unique pouch configuration according to the invention provides other distinct benefits. From a manufacturing standpoint, it is not necessary to provide a secure closure means, such as an adhesive strip or the like. The wrapper material acts as the closure device with substantially little additional manufacturing steps. Also, unlike adhesive or tape that tends to degrade and loose its effectiveness with continued use, the panel closure device of the present invention can be re-used extensively without concern regarding the integrity of the closure. This may be particularly important for purse-pack applications wherein the pouch would be opened and closed numerous times. With the present pouch configuration, the same wrapper material can be used for both individually wrapped articles and purse-pack configurations. With many conventional designs, the purse-pack requires a more “substantial” material to maintain the integrity and shape of the pouch. Also the pouch configuration maximizes the area at the open end of the pouch through which a user can insert and remove articles. Other benefits will become apparent with use of the pouch.

It is intended that the present invention include variations and modifications to the embodiments described herein as come within the scope and spirit of the invention as set forth in the appended claims.

What is claimed is:

1. An absorbent article package, comprising:
at least one absorbent article;
a wrapper material configured into a pouch having a front and back surface, closed bottom and sides, and an open top, said absorbent article carried in said pouch;
a panel disposed across said back surface, said panel having a top edge contiguous with said open top, a bottom edge, and side edges sealed to said pouch sides, said panel having a length between said top and bottom edges so as to extend less than about half-way down said pouch back surface; and
wherein to seal said absorbent article into said pouch, said panel is pulled over said open top of said pouch with said panel sides remaining sealed to said pouch sides, and to unseal said pouch to remove said absorbent article, said panel is pulled back over said open top to said back surface of said pouch.

2. The package as in claim 1, wherein said panel is formed of said wrapper material and continuous with said pouch back surface.

3. The package as in claim 2, wherein said wrapper material comprises a continuous strip of material having a first end portion folded at a first fold axis, and a second end portion folded in an opposite direction at a second fold axis, said first end portion defining said pouch front surface, said material between said first and second axis defining said pouch back surface, and said second end portion defining said panel.

4. The package as in claim 3, wherein said sides of said first portion are sealed to said sides of said back portion to define said closed pouch sides, and said sides of said panel are sealed to said pouch sides.

5. The package as in claim 3, wherein said panel extends about one-third of the way down said back surface.

6. The package as in claim 1, wherein said wrapper material comprises at least one liquid impervious film layer.

7. The package as in claim 1, wherein said wrapper material comprises a film/non-woven laminate.

8. The package as in claim 1, wherein said open top is defined by aligned ends of said back surface and said front surface.

9. An absorbent article package, comprising:
a wrapper material configured into a pouch having an open top, said pouch having an absorbent article carried therein;
a portion of said wrapper material folded at a fold axis co-extensive with said open top, said portion folded away from said open top so as to extend along a back surface of said pouch;
said portion having sides bonded to sides of said pouch and an edge unbonded to said back surface between said bonded sides.

10. The package as in claim 9, wherein said portion extends less than about half-way down said back surface.

11. The package as in claim 9, wherein said pouch has a closed bottom defined by a different fold of said wrapper material.

12. The package as in claim 11, wherein said pouch has sealed sides defined by bonded aligned sides of said wrapper material extending from said different fold to said open top.

13. The package as in claim 12, wherein said portion sides are bonded with said aligned sides of said wrapper material in a single bonding operation.
